(19) United States
(12) Patent Application Publication
(10) Pub. No.: US 2018/0311158 A1
(43) Pub. Date: Nov. 1, 2018

(54) FUNCTIONALIZED MWCNT COMPLEX STRUCTURE CONTAINING SIO2@EDAS NANO-PARTICLES AND AG@CITRATE NANO-MOLECULES AND OF HIGH MOISTURIZATION AND STERILIZATION

(71) Applicants: HONG-ZHENG LAI, Hsinchu City (CN); Li-Tien Huang, Hsinchu City (CN); I-Lin Lee, Hsinchu City (CN); Cheng-Sung Lin, Hsinchu City (CN); Tseng-Lung Chang, Hsinchu City (CN)

(72) Inventors: HONG-ZHENG LAI, Hsinchu City (CN); Li-Tien Huang, Hsinchu City (CN); I-Lin Lee, Hsinchu City (CN); Cheng-Sung Lin, Hsinchu City (CN); Tseng-Lung Chang, Hsinchu City (CN)

(21) Appl. No.: 15/499,857

(22) Filed: Apr. 27, 2017

(51) Int. Cl. A61K 9/00; A61K 33/38; A61Q 19/00; A61K 8/58; A61K 8/02; B32B 9/00

(52) U.S. Cl. CPC A61K 9/0092; A61K 33/38; A61Q 19/007; B32B 2264/102; A61K 8/0204; B32B 9/007; A61K 8/585

(57) ABSTRACT

A functionalized MWCNT complex structure containing SiO2@EDAS nano-particles and Ag@Citrate nano-molecules and of high moisturization and sterilization includes a functionalized MWCNT; carbon atoms of a benzene of the functionalized MWCNT being combined with other functional groups containing COOH, OH and SO; a plurality of SiO2@EDAS nano-particles which are formed by modifying N-[3-(2-Aminoethylamino) propyl] trimethoxysilane (EDAS) on SiO2 nano-particles; the adhering layer further containing a plurality of Ag@Citrate nano-molecules. Therefore, since the functionalized MWCNTs 10 contains both the SiO2@EDAS nano-particles 21 and the Ag@Citrate nano-molecules 22 and thus it has the functions of the moisturization and sterilization.

### FIELD OF THE INVENTION

The present invention is related to MWCNT, and in particular to a functionalized MWCNT complex structure containing SiO2@EDAS nano-particles and Ag@Citrate nano-molecules and of high moisturization and sterilization.

### BACKGROUND OF THE INVENTION

Carton Nanotube (CNT) has a tubular carbon molecule structure. Carbon atoms composed in the continuous aromatic ring is used as backbones of a CNT. Each carbon atom in the CNT is connected through a σ bonding of the sp2 hybrid orbital and a π bonding and thus the carbon atoms form a hexagonal cellular structure. All the non-hybrid P electrons (electrons in P orbital) of each carbon atom on the CNT are formed as conjugated π electron cloud on the CNT. Based on the number of the layers of the CNT, the CNT could be classified as single wall CNTs and functionalized multiple wall CNTs. The CNT is thin in the radial direction thereof. It only has a size about several nanometers, while along the axial direction, the thickness may be several tenth or hundredth micrometers.

By the property of the CNT, many different kinds of complex materials are made. In biologics, CNTs provide the function of regeneration of cells. Furthermore, it can absorb infrared rays and thus potentially available in heat treatments. Therefore, inventors of the present invention desires to apply CNTs on the biological medicine, especially, using CNTs on wound sterilization is preferred. However, these kinds of biological medical material are required to have the functions of moisturization and sterilization. The moisturization is achievable by modifying amine on silicon material for absorbing water. Sterilization is achievable by adding Ag nano-material. The original CNT has no function of moisturization and sterilization. Moreover, commercial Ag material is added to other material by coating which is unstable and the quantity thereof is improper. Some Ag may be fallen down apparently.

Therefore, the present invention provides a functionalized MWCNT complex structure containing SiO2@EDAS nano-particles and Ag@Citrate nano-molecules and of high moisturization and sterilization so as to improve the defects above mentioned prior art.

### SUMMARY OF THE INVENTION

Accordingly, the object of the present invention is to provide a functionalized MWCNT complex structure containing SiO2@EDAS nano-particles and Ag@Citrate nano-molecules and of high moisturization and sterilization. in the present invention, since the functionalized MWCNTs 10 contains both the SiO2@EDAS nano-particles 21 and the Ag@Citrate nano-molecules 22 and thus they have the functions of the moisturization and sterilization.

To achieve about object, the present invention provides a functionalized MWCNT complex structure containing SiO2@EDAS nano-particles and Ag@Citrate nano-molecules and of high moisturization and sterilization comprising functionalized MWCNT; carbon atoms of a benzene of the functionalized MWCNT being combined with other functional groups containing COOH, OH and SO; the way for obtaining the functionalized MWCNT is: to mix long MWCNT with H2SO4 and HNO3 for a predetermined time to cause that the HNO3 to oxidize the long MWCNT; by dehydration of the H2SO4; the long MWCNT generating above functional groups; during oxidization, due to chemical reaction, surface of long MWCNT generating new function groups because some defects are formed in the long MWCNT; variations and breaking of chemical bonds will affect the structures of longer MWCNT so that the length thereof is reduced and thus functionalized MWCNTs are formed.

A polarized adhering layer is adhered on the functionalized MWCNT of acylation by surface charges thereof; and the polarized layer mainly contains of: a plurality of SiO2@EDAS nano-particles which are formed by modifying N-[3-(2-Aminoethylamino) propyl] trimethoxysilane (EDAS) on SiO₂ nano-particles; electrical charges on the surface of the SiO₂@EDAS nano-particles being positive; therefore, they can be combined with the functionalized MWCNT whose surface is charged negatively so as to form a complex structure.

The adhering layer further comprises a plurality of Ag@Citrate nano-molecules which are formed by using sodium citrate as a reducing agent to reduce Ag ions and generate a great number of connection bonds so that molecules of citrate acid encloses groups of Ag atoms to form Ag@Citrate nano-molecule 22; at this structure, surfaces of nano-molecule are negatively charged; generally, sizes of Ag@Citrate nano-molecules 22 are about 10 to 20 nanometers so that the volume thereof is far smaller than that of the SiO2@EDAS nano-particles 21; the two have opposite electric polarities; therefore, Ag@Citrate nano-molecules 22 being absorbed around the surfaces of the SiO2@EDAS nano-particles 21; because the SiO2@EDAS nano-particles 21 are attached on the functionalized MWCNT 10 and thus the Ag@Citrate nano-molecules 22 are also attached on the functionalized MWCNT to form a multilayered complex structure; and wherein the Ag@Citrate nano-molecules release Ag ions which present sterilization effect so that the MWCNT with the Ag@Citrate nano-molecules has an effect of sterilization.

### BRIEF DESCRIPTION OF THE DRAWINGS

FIG. 1 is an assembly schematic view of the present invention.

FIG. 2 is another assembly view of the present invention.

FIG. 3 shows a distribution about the diameters of hydrophicity particles of SiO2.

FIG. 4 shows a distribution about the diameters of hydrophicity particles of SiO2@EDAS.

### DETAILED DESCRIPTION OF THE INVENTION

In order that those skilled in the art can further understand the present invention, a description will be provided in the following in details. However, these descriptions and the appended drawings are only used to cause those skilled in the art to understand the objects, features, and characteristics of the present invention, but not to be used to confine the scope and spirit of the present invention defined in the appended claims.

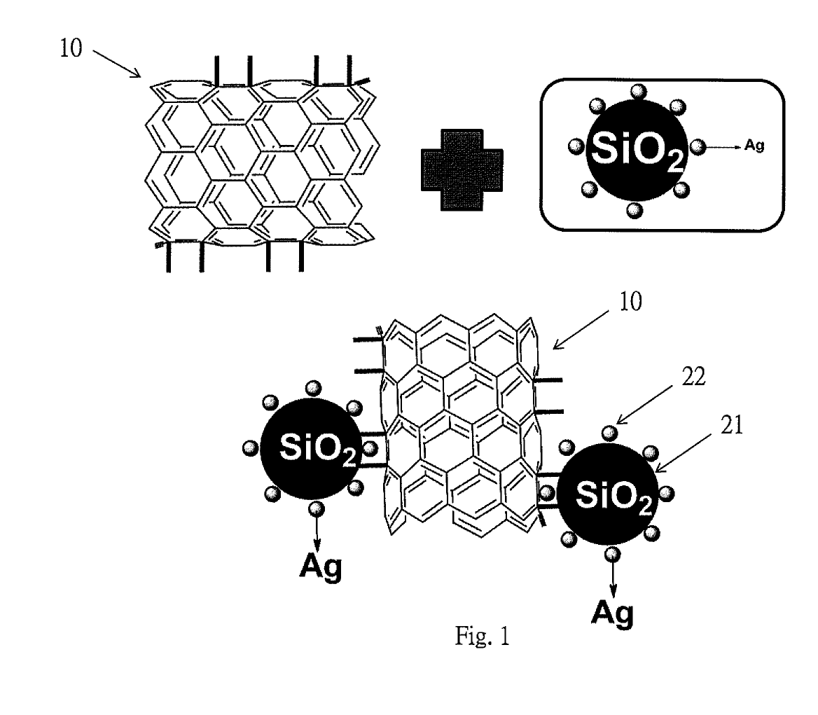

Referring to FIGS. 1 to 4, the structure of the present invention is illustrated. The present invention includes the following elements:

A functionalized MWCNT 10 has a length between 35 μm to 60 μm. The carbon atoms of the benzene of the functionalized MWCNT are combined with other functional groups, such as COOH, OH and SO. The way for obtaining the functionalized MWCNT is to mix a long MWCNT with a length of 300 μm with H₂SO₄ and HNO₃ through a predetermined time to cause that the HNO₃ to oxidize the long MWCNT. By dehydration of the H₂SO₄, the MWCNT generates above functional groups. In the process of oxidization, due to chemical reaction, surface of long MWCNT generates new function groups because some defects are formed in the structure of the long MWCNT.

Variations and breaking of chemical bonds will affect the structures of the long MWCNT so that the length thereof is reduced and thus the functionalized MWCNT is formed. The functional groups of COOH, OH and SO cause surfaces of the MWCNT are negatively charged so that the whole function groups have a nano-structure with negative charges thereon. The charges on the surface serve to abstract particles of opposite polarity so as to enhance the absorption ability of the functionalized MWCNTs 10.

A polarized adhering layer 20 adheres on the MWCNT with acylation by the surface charges thereof. The polarized layer mainly contains of:

A plurality of SiO2@EDAS nano-particles 21 which are formed by modifying N-[3-(2-Aminoethylamino) propyl] trimethoxysilane (EDAS) on the SiO2 nano-particles 21. The size of the SiO2@EDAS nano-particles 21 is about 200 nm. The electrical charges on the surface of the SiO2@EDAS nano-particles 21 are positive. Therefore they can be combined with the functionalized MWCNT 10 whose surface is charged negatively to form a complex structure.

A surface of the SiO2@EDAS nano-particle 21 has NH2 bones which can form H-bonds with the hydrogen ions of water (H2O) so as to firmly secure water molecules thereon. Furthermore, by the hydrophilic property of the material, the SiO2@EDAS nano-particles 21 can firmly secure to the water molecules to have the effect of moisturization.

The adhering layer 20 further comprises a plurality of Ag@Citrate nano-molecules 22 which are formed by: using sodium citrate as a reducing agent to reduce Ag ions and generates a great number of connection bonds so that molecules of citrate acid encloses groups of Ag atoms to form Ag@Citrate nano-molecules 22. At this structure, surfaces of nano-molecule is negatively charged. As illustrated in FIG. 1, generally, sizes of Ag@Citrate nano-molecules 22 are about 10 to 20 nanometers so that the volume thereof is far smaller than that of the SiO2@EDAS nano-particles 21. The two have opposite electric polarities. Therefore Ag Citrate nano-molecules 22 are absorbed around the surfaces of the SiO2@EDAS nano-particles 21. Because the SiO2@EDAS nano-particles 21 are attached on the functionalized MWCNT 10 and thus the Ag@Citrate nano-molecules 22 are also attached on the functionalized MWCNT 10 so as to form a multilayered complex structure.

The Ag@Citrate nano-molecules will release Ag ions which has preferred sterilization effect so that the MWCNTs 10 with the Ag@Citrate nano-molecules have the effect of sterilization.

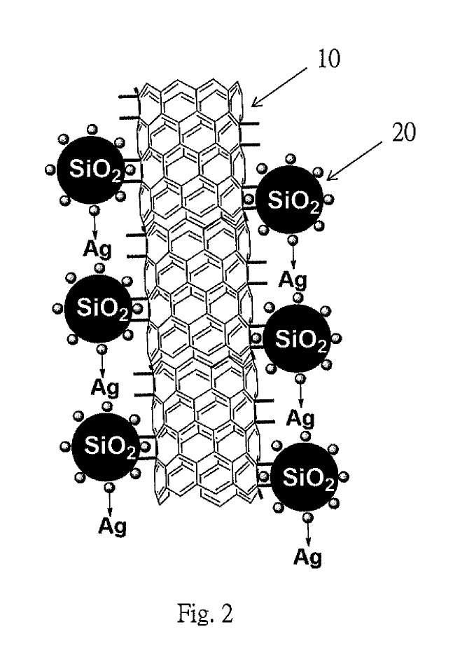

By above mentioned structure, as illustrated in FIG. 2, since the Ag@Citrate nano-molecules 22 are absorbed around and electrically attractive with the SiO2@EDAS nano-particles 21, as a result, the Ag@Citrate nano-molecules 22 are also arranged around the functionalized MWCNTs 10.

If no SiO2@EDAS nano-particle 21 is used, it is impossible to attract the Ag@Citrate nano-molecules 22 around the functionalized MWCNTs 10. Therefore, in the present invention, since the functionalized MWCNTs 10 contains both the SiO2@EDAS nano-particles 21 and the Ag@Citrate nano-molecules 22 and thus they have the functions of the moisturization and sterilization.

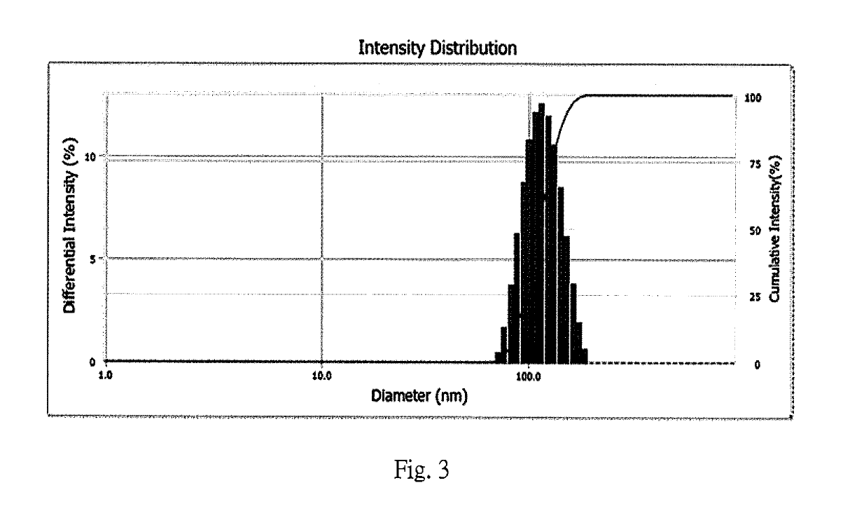

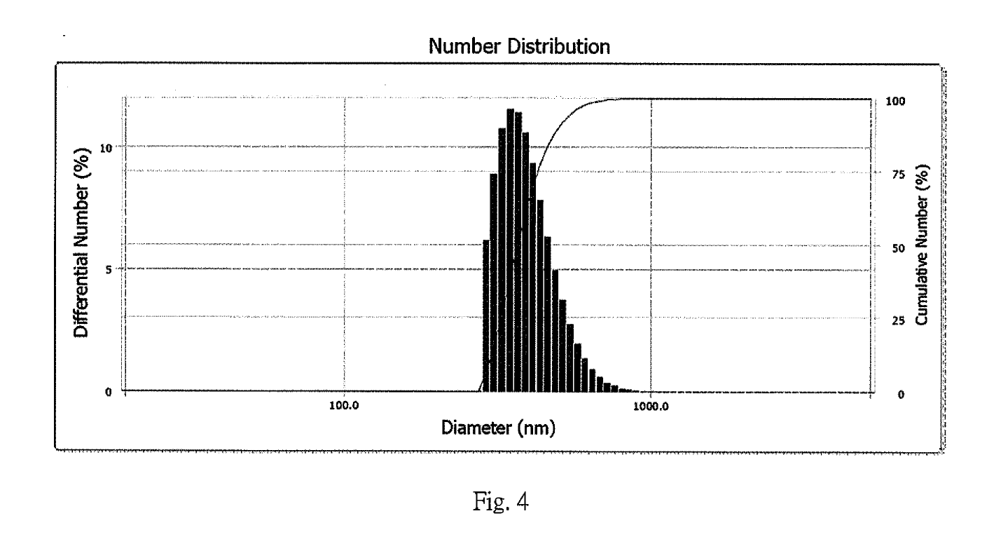

The effects of the present invention are shown in FIGS. 3 and 4, which are obtained from experiments. FIG. 3 shows radius of the SiO2 hydrophicity particles, in that the average of the hydrophicity particles is 126 nm. FIG. 4 shows that after hydrophicity of SiO2@EDAS, the average diameters of the hydrophicity particles are about 400 nm. In the present invention, the sizes of the SiO2 is about 120˜160 nm. The SiO2@EDAS after adding EDASs has sizes about 110-200 nm. Therefore, it is apparent that the water absorption of SiO2 is not good due to small hydrophicity radius. While after hydrophicity, the SiO2@EDAS has a size of about 400 nm. This is because the functional groups around the SiO2@EDAS have preferred water attraction property and thus hydrophicity radius increases greatly. Moreover, from experiment, the upper and lower surfaces of the functionalized MWCNTs are charged negatively apparently.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A functionalized MWCNT complex structure containing SiO2@EDAS nano-particles and Ag@Citrate nano-molecules and of high moisturization and sterilization, comprising:
a functionalized MWCNT; carbon atoms of a benzene of the functionalized MWCNT being combined with other functional groups containing COOH, OH and SO; the way for obtaining the functionalized MWCNT is: to mix long MWCNT with H₂SO₄ and HNO₃ for a predetermined time to cause that the HNO₃ to oxidize the long MWCNT; by dehydration of the H₂SO₄; the long MWCNT generating above functional groups; during oxidization, due to chemical reaction, surface of long MWCNT generating new function groups because some defects are formed in the long MWCNT; variations and breaking of chemical bonds will affect the structures of longer MWCNT so that the length thereof is reduced and thus functionalized MWCNTs are formed;
wherein the functional groups of COOH, OH and SO cause surfaces of the MWCNT are negatively charged so that the whole function groups have a nano-structure with negative charges thereon; the charges on the surface serves to absorb particles of opposite polarity so as to enhance the absorption ability of the functionalized MWCNTs.

2. The functionalized MWCNT complex structure as claimed in claim 1, further comprising
a polarized adhering layer adhering on the functionalized MWCNT of acylation by surface charges thereof; and the polarized layer mainly contains of:
a plurality of SiO₂@EDAS nano-particles which are formed by modifying N-[3-(2-Aminoethylamino) propyl] trimethoxysilane (EDAS) on SiO₂ nano-particles; electrical charges on the surface of the SiO₂@EDAS nano-particles being positive; therefore, they can be combined with the functionalized MWCNT whose surface is charged negatively so as to form a complex structure;
wherein surfaces of the SiO₂@EDAS nano-particles having NH₂ bones can form H-bonds with hydrogen ions of water (H₂O) so as to firmly secure water molecules thereon; further, by the hydrophilic property of the material, the SiO₂@EDAS nano-particles firmly secure to the water molecules to have an effect of moisturization.

3. The functionalized MWCNT complex structure as claimed in claim 2, wherein
the adhering layer further comprising a plurality of Ag@Citrate nano-molecules which are formed by using sodium citrate as a reducing agent to reduce Ag ions and generate a great number of connection bonds so that molecules of citrate acid encloses groups of Ag atoms to form Ag@Citrate nano-molecule; at this structure, surfaces of nano-molecule are negatively charged; generally, sizes of Ag@Citrate nano-molecules are about 10 to 20 nanometers so that the volume thereof is far smaller than that of the SiO2@EDAS nano-particles; the two have opposite electric polarities; therefore, Ag@Citrate nano-molecules being absorbed around the surfaces of the SiO₂@EDAS nano-particles; because the SiO₂@EDAS nano-particles are attached on the functionalized MWCNT 10 and thus the Ag@Citrate nano-molecules are also attached on the functionalized MWCNT to form a multilayered complex structure; and
wherein the Ag@Citrate nano-molecules release Ag ions which present sterilization effect so that the MWCNT with the Ag@Citrate nano-molecules has an effect of sterilization.

4. The functionalized MWCNT complex structure as claimed in claim 1, wherein the functionalized MWCNT has a length between 35 μm to 60 μm.

5. The functionalized MWCNT complex structure as claimed in claim 2, wherein a size of the SiO2@EDAS nano-particle is about 200 nm.

6. The functionalized MWCNT complex structure as claimed in claim 3, wherein a size of the Ag@Citrate nano-molecule is from 10 to 20 nano-meters.
